(19) United States
(12) Patent Application Publication
(10) Pub. No.: US 2018/0093107 A1
(43) Pub. Date: Apr. 5, 2018

(54) System and Method For Healing and/or Disinfecting Wounds and Burns

(71) Applicant: Gliese 623B, LLC, Mendon, MA (US)

(72) Inventor: Anthony Ball, Pepperell, MA (US)

(21) Appl. No.: 15/718,952

(22) Filed: Sep. 28, 2017

Related U.S. Application Data
(60) Provisional application No. 62/401,975, filed on Sep. 30, 2016.

(51) Int. Cl. A61N 5/06

(52) U.S. Cl. CPC A61N 5/0616; A61N 5/0624; A61N 2005/0626; A61N 2005/0663; A61N 2005/0661

(57) ABSTRACT

A system for healing and/or disinfecting wounds and burns includes an emitter including one or more blue light sources configured to emit blue light at a therapeutic energy level at a wound or burn area of a human or animal subject and one or more ultraviolet-A (UVA) light sources configured to emit UVA light at a therapeutic energy level at the wound or burn area. A controller coupled to the emitter is configured to control the therapeutic energy level of the blue light and the UVA light and one or more parameters associated with the blue light and the UVA light to produce a photobiomodulation effect in order to induce a cytokine response in cells of the wound or burn area to elicit recruitment and proliferation of cells to promote healing of the wound or burn area.

### RELATED APPLICATIONS

This application claims benefit of and priority to U.S. Provisional Application Ser. No. 62/401,975 filed Sep. 30, 2016, under 35 U.S.C. §§ 119, 120, 363, 365, and 37 C.F.R. § 1.55 and § 1.78, which is incorporated herein by this reference.

### FIELD OF THE INVENTION

This invention relates to a system and method for healing and/or disinfecting wounds and burns.

### BACKGROUND OF THE INVENTION

The emergence and re-emergence of antimicrobial resistance leads to an enormous clinical burden which may result in death of millions of people and a tremendous socioeconomic burden globally each year. Multidrug and Pandrug resistance microorganisms include a class of elite pathogens with enhanced virulence and pathogenicity traits. For example, Methicillin resistance Staphylococci aureus (MRSA) in the U.S. alone is responsible for nearly 20,000 deaths per year and carries an estimated 3 to 4 billion dollars in added healthcare costs. Individuals seeking medical treatment in a hospital often acquire a hospital acquired infection (HAIs), as some hospitals have become notorious repositories harboring extremely pathogenic and drug-resistant microbes. Additionally, surgical wounds and sores are common sites for opportunistic microbes. Additionally, surgical wounds and sores are common sites for opportunistic pathogens to establish life threatening infections. Burns are equally at risk, but are particularly problematic because they can affect large areas of the epidermis depending on the degree of the burned skin. One of the major reasons that open wounds and burns are prone to infections may be attributed to the function of intact skin to provide a barrier to the entry of microorganisms. Certain individuals, such as diabetics, the elderly, or immune-compromised patients, and the like, are especially at risk due to an impaired ability to heal.

Moreover, it has been established that skin pigmentation, which is caused by the presence and accumulation of endogenous chromophores within the pathogen, may be associated with virulence. One classification of pigments found in pathogens is porphyrin. Light energy within the visible, e.g., blue light and red light, and the ultraviolet spectrum has been proven effective in eliciting of microbial pathogen eradication at some level. The effect of light to eradicate pathogens correlates and is dependent upon the presence of oxygen. The photoexcitation of endogenous chromophores, including porphyrins, leads to the production of reactive oxygen species (ROS), such as hydroxyl radicals, superoxides, peroxides, and singlet oxygen, which elicit killing of microbial cells. Under normal conditions, pigmentation confers a competitive advantage for the pathogen because the pigments are antioxidants which help to protect the pathogen from destruction by the host immune system.

UVA light does not occur alone in the environment. UVA light is always present with ultraviolet-B (UVB), all of which enter the atmosphere and are generated by the sun About 90% of all photodamage to human cells is associated with UVB light which cross-links DNA in leading to mutagenesis in the cell. UVA light, unlike UVB light, is generally well-tolerated by cells because it is weakly absorbed by DNA. This is because UVB directly damaging DNA, whereas UVA excites endogenous chromophores, leading to the expression of ROS.

Collagen is the main structural component of skin. IFN-C is an interferon. Interferons are members of a class of chemicals known as cytokines. Cytokines play a key role in cellular communication and serve to prime the immune system which aids in the clearance of foreign antigens such as those expressed by bacteria or mutagenized cells in the case of some cancers. Cytokines can initiate cellular repair.

UV light (with both UVA and UVB) has been proposed as a potential modulator of keratinocyte-melanocyte cross talk in promoting wound healing. Keratinocytes, the main cell type in the epidermis produce collagen, form a self-renewing epithelial barrier to protect the skin against environmental hazards, while melanocytes, located in the basal layer of the epidermis, are dendritic-like pigment-producing cells, which protect keratinocytes against the DNA-damaging effects of UVB irradiation through production of melanin.

Thus, there is a need for an effective system and method for healing and/or disinfecting wounds and burns that utilizes visible light (blue and red) and only UVA light which when applied properly to a wound or burn area elicits a cellular response in humans and animals to produce phototoxic byproducts that kills pathogens to disinfect the wound or burn area and initiates a repair response which leads to cellular proliferation and regrowth of the affected tissue to promote healing of the wound or burn area.

### SUMMARY OF THE INVENTION

In one aspect, a system for healing and/or disinfecting wounds and burns, the system includes an emitter including one or more blue light sources configured to emit blue light at a therapeutic energy level at a wound or burn area of a human or animal subject and one or more ultraviolet-A (UVA) light sources configured to emit UVA light at a therapeutic energy level at the wound or burn area. A controller coupled to the emitter is configured to control the therapeutic energy level of the blue light and the UVA light and one or more parameters associated with the blue light and the UVA light to produce a photobiomodulation effect in order to induce a cytokine response in cells of the wound or burn area to elicit recruitment and proliferation of cells to promote healing of the wound or burn area.

In one embodiment, the controller may be configured to control the therapeutic energy level of the blue light and the UVA light and one or more parameters associated with the blue light and the UVA light to activate photo excitation of intracellular accumulated chromophores and production of cytotoxic reactive oxygen species in opportunistic pathogens such that a synergistic effect of the combination of the blue light at the therapeutic energy level and the UVA light at the therapeutic energy level and the one or more parameters associated with the blue light and the UVA light kills opportunistic pathogens in the wound or burn area to disinfect an infected wound area or an infected burn area. The therapeutic energy level of the blue light and the UVA light may be in the range of about 0.4 J/cm² to about 4 J/cm². The one or more parameters of the blue light and the UVA light controlled by the controller may include one or more of: a wavelength of the blue light, a wavelength of the UVA light, a frequency of the blue light, a frequency of the UVA light, one or more waveforms of the blue light, one or more waveforms of the UVA light, one or more duty cycles of the blue light, and/or one or more duty cycles of the UVA light. The wavelength of the blue light may be in the range of about 405 nm to about 470 nm. The wavelength of the UVA light may be in the range of about 315 nm to about 400 nm. The frequency of the blue light may be in the range of about 0.5 Hz to about 1,000 Hz. The frequency of the UVA light may be in the range of about 0.5 Hz to about 1,000 Hz. The one or more waveforms may include one or more of a sinewave, a square wave, a triangle wave, and/or a sawtooth wave. The controller may be configured to control the one or more blue light sources and the one or more UVA light sources to emit the blue light and the UVA light as pulsed light. The controller may be configured to control the one or more blue light sources and the one or more UVA light sources to emit the blue light and the UVA light as continuous light. The controller may be configured to control the one or more blue light sources and the one or more UVA light sources the blue light and the UVA light in a combination of pulsed light and continuous light. The one or more duty cycles of the blue light may include one or more of: a 25% duty cycle, a 50% duty cycle, or a 75% duty cycle. The one or more duty cycles of the UVA light may include one or more of: a 25% duty cycle, a 50% duty cycle, or a 75% duty cycle. The one or more blue light sources and the one or more UVA light sources may be configured as a multi-emitter array. The controller may be configured to control the multi-emitter array to provide the blue light at the therapeutic energy level and the UVA light at the therapeutic energy level and at the one or more parameters associated with the blue light and the UVA light. The controller may be configured to shut off the one or more blue light sources and the one or more UVA sources when a predetermined therapeutic dosage of blue light and UVA light is applied to the wound or burn area. The emitter may include one or more red light sources configured to emit red light at a therapeutic energy level at the wound or burn area. The controller may be coupled to the one or more red light source and is configured to control the therapeutic energy level of the red light and one or more parameters associated with the red light to increase oxygenation, vascularization and recruitment of immune cells in the wound or burn area and to enhance the photoexcitation of accumulated intracellular chromophores and production of cytotoxic reactive oxygen species in opportunistic pathogens. The one or more blue light sources, the one or more UVA light sources, and the one or more red light sources may be configured as a multi-emitter array. The controller may be configured to control the multi-emitter array to provide the blue light at the therapeutic energy level, the UVA light at the therapeutic energy level, and the red light at the therapeutic energy level, and one or more parameters associated with the blue light, the UVA light, and the red light. The controller may be responsive to a wound or burn area detection device and is configured to determine a size of the wound or burn area. The controller may be configured to provide the therapeutic energy level of the blue light, the therapeutic energy level of the UVA light, and the one or more parameters associated with the blue light and the UVA light by controlling the power applied to the emitter. The system may include a display device coupled to the controller.

In another aspect, a system for healing and/or disinfecting wounds and burns is featured. The system includes an emitter including one or more blue light sources configured to emit blue light at a therapeutic energy level at a wound or burn area of a human or animal subject one or more (ultraviolet) UVA light sources configured to emit UVA light at a therapeutic energy level at the wound or burn area. A controller coupled to the one or more blue light sources and the one or more UVA light sources is configured to control the therapeutic energy level of the blue light and the UVA light and one or more parameters associated with the blue light and the UVA light to activate photoexcitation of intracellular accumulated chromophores in the production of cytotoxic reactive oxygen species in opportunistic pathogens such that a synergistic effect of the combination of the blue light at the therapeutic energy level and the UVA light at the therapeutic energy level and the UVA light at the therapeutic energy level and the one or more parameters associated with the blue light and the UVA light kills the opportunistic pathogens in the wound or burn area to disinfect an infected wound or an infected burn area.

In another aspect, a system for healing and/or disinfecting wounds and burns is featured. The system includes an emitter including one or more blue light sources configured to emit blue light at a therapeutic energy level at a wound or burn area of a human or animal subject and one or more (ultraviolet) UVA light sources configured to emit UVA light at a therapeutic energy level at the wound or burn area. A controller coupled to the one or more blue light sources and the one or more UVA light sources is configured to control the therapeutic energy level of the blue light and the UVA light and one or more parameters associated with the blue light and the UVA light to produce a photobiomodulation effect in order to induce a cytokine response in cells of the wound or burn area to elicit recruitment and proliferation of cells to promote healing of the wound or burn area and to activate photoexcitation of intracellular accumulated chromophores and production of cytotoxic reactive oxygen species in opportunistic pathogens such that a synergistic effect of the combination of blue light at the therapeutic energy level and UVA light at the therapeutic energy level and the UVA light at the therapeutic energy level and the one or more parameters associated with the blue light and the UVA light kills the opportunistic pathogens in the wound or burn area to disinfect an infected wound area or an infected burn area.

In yet another aspect, a method for healing and/or disinfecting wounds and burns is featured. The method includes applying blue light at a therapeutic energy level at a wound or burn area of a human or animal subject, applying ultraviolet-A (UVA) light at a therapeutic energy level at the wound or burn area, and controlling the therapeutic energy level of the blue light and the UVA light and one or more parameters associated with the blue light and the UVA light to produce a photobiomodulation effect in order to induce a cyctokine response in the cells of the wound or burn area to illicit recruitment and proliferation of cells to promote healing of the wound or burn area.

In one embodiment, controlling the therapeutic energy level of the blue light and the UVA light and the one or more parameters associated with the blue light and the UVA light may activate photoexcitation of intracellular accumulated chromophores and the production of cytotoxic reactive oxygen species in the opportunistic pathogens such that a synergistic effect of the combination of the blue light at the therapeutic energy level and the UVA light at the therapeutic energy level and the UVA light at the therapeutic energy level and the one or more parameters associated with the blue light and the UVA light kills opportunistic pathogens in the wound or burn area to disinfect an infected wound area or an infected burn area. The one or more parameters of the blue light and the UVA light may include controlling one or more of: a wavelength of the blue light, a wavelength of the UVA light, a frequency of the blue light, a frequency of the UVA light, one or more waveforms of the blue light, one or more waveforms of the UVA light, one or more duty cycles of the blue light, and one or more duty cycles of the UVA light. The blue light and the UVA light may be applied at a therapeutic energy level in the range of about 0.4 J/cm² to about 4 J/cm². The blue light may be applied at a wavelength in the range of about 405 nm to about 470 nm. The UVA light may be applied at a wavelength in the range of about 315 nm to about 400 nm. The one or more waveforms may include a sinewave, square wave, a triangle wave, and/or a sawtooth wave. The blue light may be applied at a frequency in the range of about 0.5 Hz to about 1,000 Hz. The UVA light may be applied at a frequency in the range of about 0.5 Hz to about 1,000 Hz. The one or more duty cycles of the blue light may include one or more of: 25%, 50%, or 75% duty cycle. The one or more duty cycles of the UVA light may include one or more of: 25%, 50%, or 75% duty cycle. The method may include applying red light at a therapeutic energy level and one or more parameters associated with the red light at the wound or burn area. The method may include applying red light at a therapeutic energy level and one or more parameters associated with the red light at the wound or burn area to increase vascularization and recruitment of immune cells of the wound area and oxygenation in the cells of the wound or burn area to enhance the photobiomodulation and/or the photoexcitation of intracellular chromophores and production of cytotoxic reactive oxygen species in opportunistic pathogens. The therapeutic energy level of the blue light, the therapeutic energy level of the UVA light, and the one or more parameters associated with the one blue light and the UVA light may be controlled by adjusting a power level applied to one or more blue light sources and one or more UVA light sources.

The subject invention, however, in other embodiments, need not achieve all these objectives and the claims hereof should not be limited to structures or methods capable of achieving these objectives.

### BRIEF DESCRIPTION OF THE SEVERAL VIEWS OF THE DRAWINGS

Other objects, features and advantages will occur to those skilled in the art from the following description of a preferred embodiment and the accompanying drawings, in which:

FIG. 1 is a schematic block diagram showing the primary components of one embodiment of the system for healing and/or disinfecting wounds and burns of this invention;

FIG. 2 is a three-dimensional view of the system shown in FIG. 1;

FIG. 3 is a three-dimensional view of the system shown in FIGS. 1 and 2 mounted on a stand;

FIG. 4 is show examples of various waveforms of light which may emitted by the one or more blue light sources and the one or more UVA light sources shown in one or more of FIGS. 1-3;

FIG. 5 is a three-dimensional view showing an example of the multi-emitter array shown in FIG. 2 positioned proximate a wound or burn area on the foot of a human subject;

FIG. 6 is a schematic block diagram showing the primary components of another embodiment of the system for healing and/or disinfecting wounds and burns of this invention;

FIG. 7 is block diagram showing one example of the primary steps of the method for healing and/or disinfecting wounds and burns of this invention;

FIG. 8 is a schematic block diagram showing an example of the process of photoinactivation by photoexcitation of endogenous chromophores by UV light 20; and

FIG. 9 shows graphs comparing the reduced energy level of blue light and UVA light provided by the synergistic effect of blue light and UVA light of the system and method shown in one or more of FIGS. 1-8 to conventional light treatment.

### DETAILED DESCRIPTION OF THE INVENTION

Aside from the preferred embodiment or embodiments disclosed below, this invention is capable of other embodiments and of being practiced or being carried out in various ways. Thus, it is to be understood that the invention is not limited in its application to the details of construction and the arrangements of components set forth in the following description or illustrated in the drawings. If only one embodiment is described herein, the claims hereof are not to be limited to that embodiment. Moreover, the claims hereof are not to be read restrictively unless there is clear and convincing evidence manifesting a certain exclusion, restriction, or disclaimer.

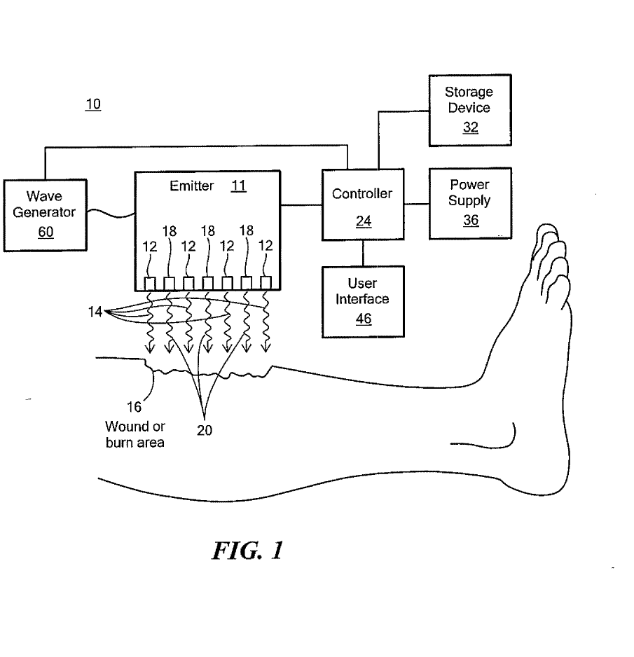

There is shown in FIG. 1, one embodiment of system 10 and the method thereof for healing and/or disinfecting wounds and burns. System 10 includes emitter 11 which includes one or more blue light sources, exemplarily indicated at 12, configured to emit blue light 14 at a therapeutic energy level at a wound or burn area of a human or animal subject, e.g., wound or burn area 16. In this example, wound or burn area 16 is located on a leg of a human subject as shown. In other examples, wound or burn area 16 may be any area of a human or animal subject has a wound area, e.g., a diabetic foot ulcer or any wound area caused by disease or medical condition, e.g., diabetic mellitus, hypertension, hyperlipidemia, arthrosclerosis, AIDS, malignancy, morbid obesity, hepatitis C virus, or any other disease or medical condition that creates a wound on a human subject or animal, or an event resulting in a contusion, hematomas, crush injury, abrasions, lacerations, incisions, punctures, or any other penetrating type wound or burn resulting from a fire, hot liquid, steam, hot metal, glass or other objects, electrical currents, radiation from X-rays, radiation therapy, sunlight or ultraviolet light from a sunlamp or tanning bed, or damaging chemicals or agents, such as strong acids, lye, paint thinner, gasoline, and the like, or a burn area.

In one design, the therapeutic energy level of blue light 14 is preferably in the range of about 0.4 J/cm² to about 4 J/cm², although the therapeutic energy level may be higher or lower than this range, but is preferably low enough to not damage the cells of wound or burn area 16 and high enough to effectively heal and/or disinfect an infected wound or burn area 16. Preferably, the wavelength of blue light 14 is in the range of about 405 nm to about 470 nm.

Emitter 11 also includes one or more UVA light sources 18 configured to emit UVA light 20 at a therapeutic energy level at wound or burn area 16. In one design, the therapeutic energy level of UVA light 20 is preferably in the range of about 0.4 J/cm² to about 4 J/cm², although the therapeutic energy level may be higher or lower than this range, but is preferably low enough to not damage the cells of wound or burn area 16 effectively heal and/or disinfect an infected wound or burn area 16. Preferably, the wavelength of UVA light 20 is in the range of about in the range of about 315 nm to about 400 nm.

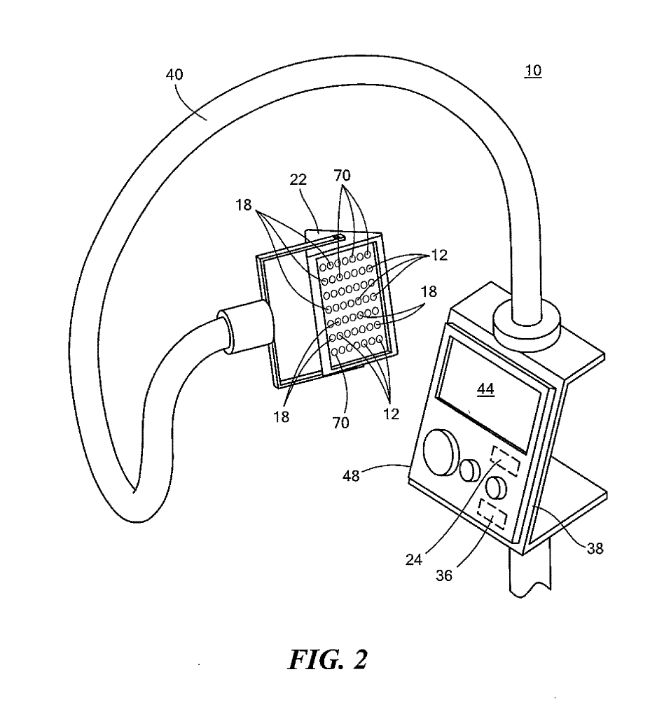

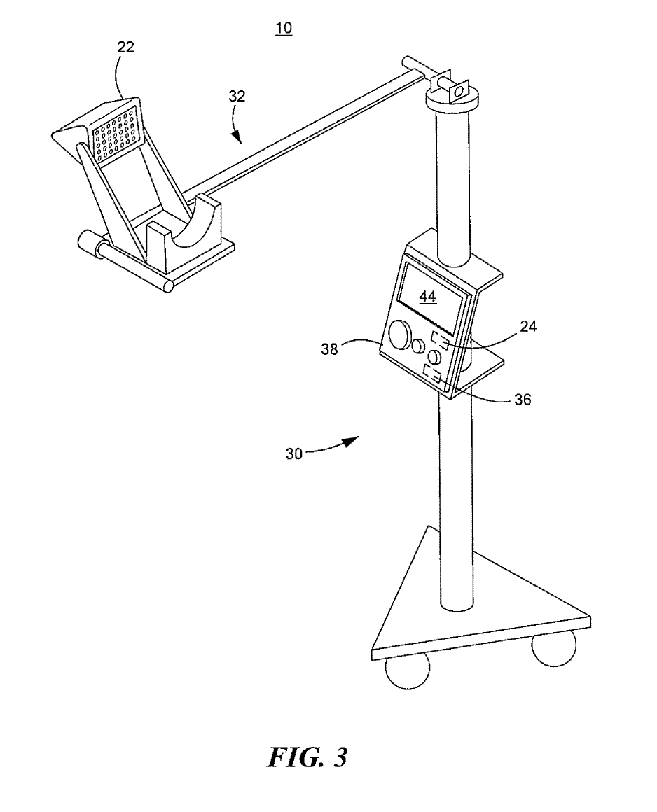

In one example, one or more blue light sources 12 and the one or more UVA light sources 18 may be light emitting diodes (LEDs), beam collimation devices, beam shaping devices, or a beam sweeping/painting device, or similar type light source. In one design, one or more blue light sources 12 and one or more UVA light sources 18 may be configured as a multi-emitter array, e.g., multi-emitter array 22, FIG. 2, where like parts have been given like numbers. FIG. 3 shows an example of system 10 configured on stand 30 having swivel arm 32 coupled to multi-emitter array 22.

System 10, FIG. 1, also includes controller 24 coupled to emitter 11, FIG. 1, or multi-emitter array 22, FIG. 2, configured to variably control the therapeutic energy level of blue light 14, the therapeutic energy level of UVA light 20, and one or more parameters associated with the blue light 14 and the UVA light 20 such that blue light 14 and UVA light 20 produce a photobiomodulation effect to induce a cytokine response in the cells of wound or burn area 16 to illicit recruitment and proliferation of cells to promote healing of wound or burn area 16, as discussed in further detail below. Controller 24 is also preferably configured to control the therapeutic energy level of the blue light 14 and the UVA light 16 and the one or more parameters associated with the blue light and the UVA light such that blue light 14 and UVA light 20 activate photoexcitation of intracellular accumulation of chromophores and production of cytotoxic reactive oxygen species (ROS) and opportunistic pathogens such that a synergistic effect of the combination of the blue light and the UVA light at their respective therapeutic energy levels and the one or more parameters associated with the blue light and the UVA light kills opportunistic pathogens in wound or burn area 16.

Controller 24 may be a processor, one or more processors, an application-specific integrated circuit (ASIC), firmware, hardware, and/or software (including firmware, resident software, micro-code, and the like) or a combination of both hardware and software that may all generally be referred to herein as a “controller”, “module”, “engine” or “system” which may be part of controller 24 or system 10. Computer program code for the programs for carrying out the instructions or operation of one or more embodiments system 10 and method for healing and/or disinfecting wounds and burns may be written in any combination of one or more programming languages, including an object oriented programming language, e.g., C++, Smalltalk, Java, and the like, or conventional procedural programming languages, such as the “C” programming language or similar programming languages or in assembly code and may be integrated or separate from processor 24. Controller 24 may be a programmable integrated circuit board.

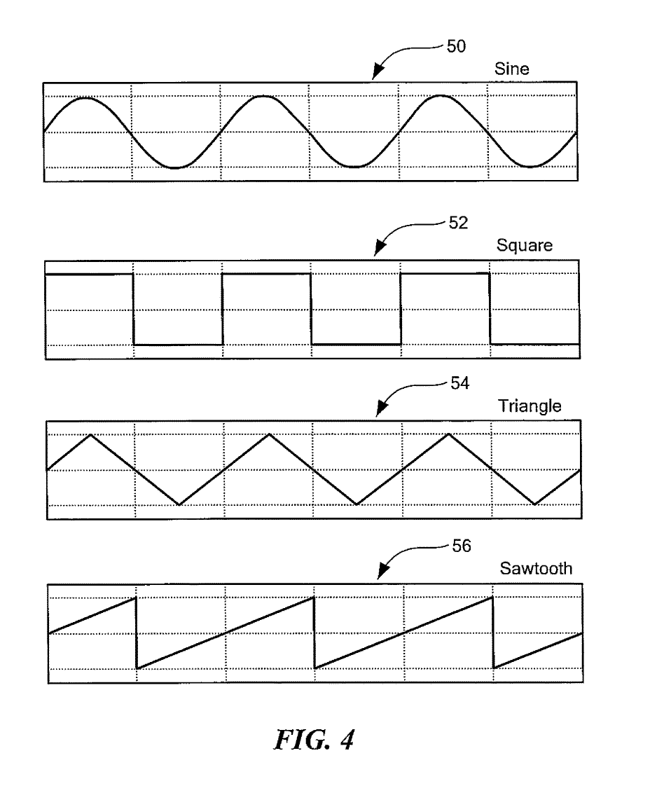

Data for controller 24 may be stored in storage device 32, FIGS. 1 and 4. Storage device 32 may include any combination of computer-readable media or memory. The computer-readable media or memory may be a computer-readable signal medium or a computer-readable storage medium. A computer-readable storage medium or memory may be, an electronic, magnetic, optical, electromagnetic, infrared, or semiconductor system, apparatus, or device, or any suitable combination of the foregoing. Other examples may include an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. As disclosed herein, the computer-readable storage medium or memory may be any tangible medium that can contain, or store one or more programs for use by or in connection with one or more processors on a company device such as a computer, a tablet, a cell phone, a smart device, or similar type device.

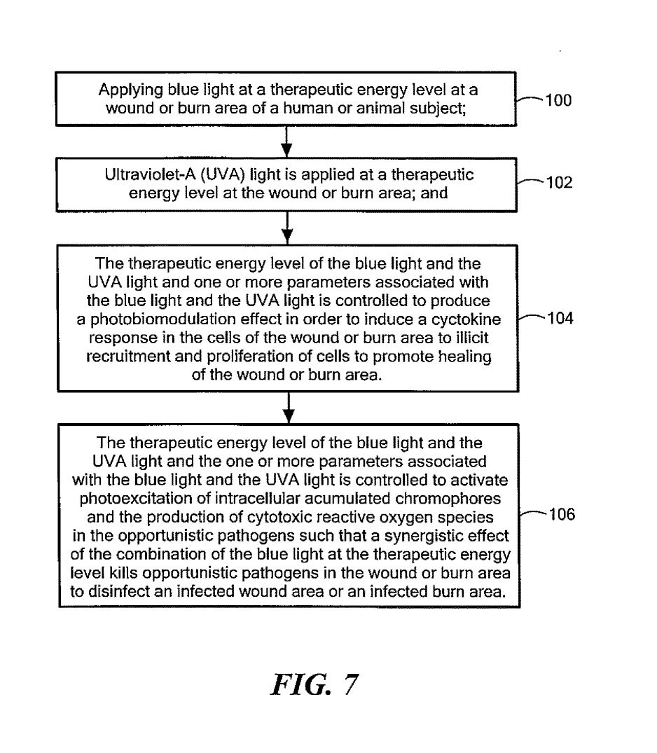

System 10, FIGS. 1-3, also preferably includes power supply 36 coupled to controller 24. In the example shown in FIGS. 2 and 3, controller 24 and power supply 36 (shown in phantom) are enclosed in housing 38 and coupled to one or more blue light sources 12 and one or more UVA light sources configured as multi-emitter array 22 by line 40. System 10 may also include display 44 which may display the total amount of energy delivered to wound or burn area 16 in a single treatment or over the course of all treatments, and the like. System 10 also preferably includes user interface 46 coupled to controller 24 which is configured to allow a user of system 10 to input the desired therapeutic energy level of the blue light 14 and the VA light 20 and the one or more parameters associated with blue light 14 and UVA light 20. In one example, user interface 46 may include control knobs 48, FIG. 7.

Controller 24 preferably controls and provides the therapeutic energy level of the blue light (0.4 J/cm² to about 4 J/cm²) and the therapeutic energy level of the UVA light (0.4 J/cm² to about 4 J/cm²) and one or more of the parameters associated with one or more blue light sources 12 and one or more UVA light sources 18 by controlling the power applied by power supply 36 to emitter 11 having one or more blue light sources 12 and the one or more UVA light sources.

The one or more parameters of blue light 14 and UVA light 20 controlled by controller 24 preferably include one or more of the wavelength of blue light 14, the wavelength of UVA light 20, the frequency of the blue light, e.g., about 0.5 Hz to about 1,000 Hz, the frequency of the UVA light, e.g., about 0.5 Hz to about 1,000 Hz, and one or more duty cycles of the blue light 14 and UVA light 20 (when blue light 14 and UVA light 20 are pulsed as discussed below), e.g., a 25% duty cycle, a 50% duty cycle, or a 75% duty cycle. The one or more parameters controlled by controller 24 also include one or more waveforms of blue light 14 and UVA light 20, e.g., sinewave 50, FIG. 4, square wave 52, triangle wave 54, or sawtooth wave 56, or similar type waveform. In one example, wave generator 60, FIG. 1, coupled to emitter 11 and controller 24 may be utilized to create the various waveforms of the therapeutic energy level of blue light 14 and UVA light 20 and the one or more parameters associated with blue light 14 and UVA light 20, discussed above, as known by those skilled in the art.

In one example, controller 24 is configured to enable one or more blue light sources 12 and one or more UVA light sources 18 to emit blue light 14 and UVA light as continuous light, pulsed light, or a combination of pulsed light and continuous light. When blue light 14 and UVA light 20 is pulsed, controller 24 may control the one or more duty cycles of the pulsed light discussed above.

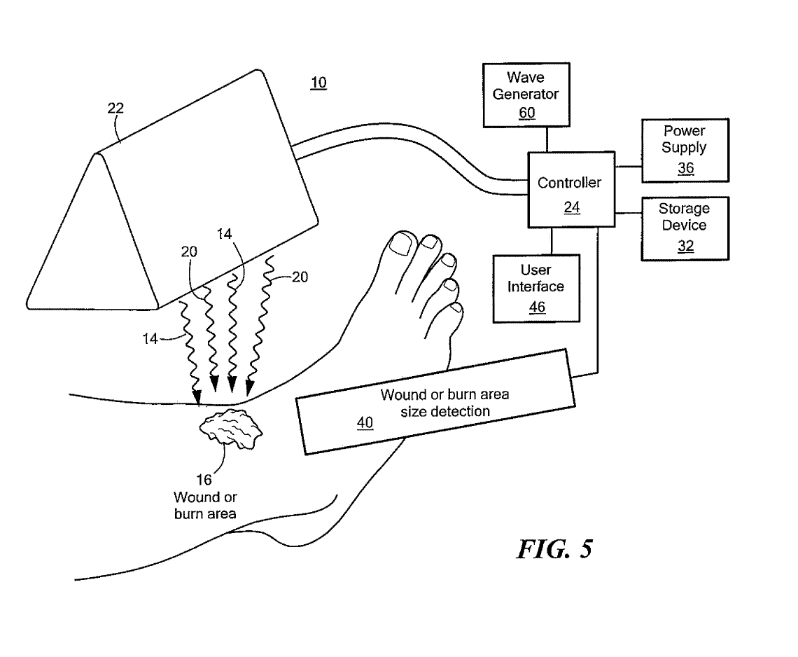

FIG. 5, where like parts have been given like numbers, shows an example of system 10 with multi-emitter array 22 placed proximate and above wound or burn area 16, in this example a foot of a human subject. In this example, multi-emitter array 22 is emitting blue light 14 at the therapeutic energy level and at one or more parameters associated with and blue light 14 as discussed above and UVA light 20 at the therapeutic energy level and at one or more parameters associated with and UVA light 20 as discussed above at wound or burn area 16 to produce a photomodulation effect in order to induce a cytokine response in the cells of a wound or burn area to illicit recruitment and proliferation of cells to promote healing of the wound or burn area 16 and/or to activate photoexcitation of intracellular accumulated chromophores and production of cytotoxic ROS and opportunistic pathogens such that a synergistic effect of the combination of blue light and UVA light at the respective energy levels kills opportunistic pathogens in wound or burn area 16 to disinfect an infected wound or burn area 16.

In this example, system 10 also preferably includes wound or burn area size detection device 40, e.g., a CCD camera or similar type device which, in combination with imaging software utilized by controller 24, detects and determines the size of wound or burn area 16 such that controller 12 can calculate or determine the required therapeutic energy level and one or more parameters associated with blue light 14 and the therapeutic energy level of UVA light 20 and the one or more parameters associated with UVA light to be delivered for healing and/or disinfecting wound or burn area 16.

In one design, controller 24 is configured to shut off one or more blue light sources 12 and one or more UVA light sources 18 when a predetermined therapeutic dosage of blue light 14 and UVA light 20 is applied to wound or burn area 16, e.g., 4 J/cm², 10 J/cm² 20 J/cm².

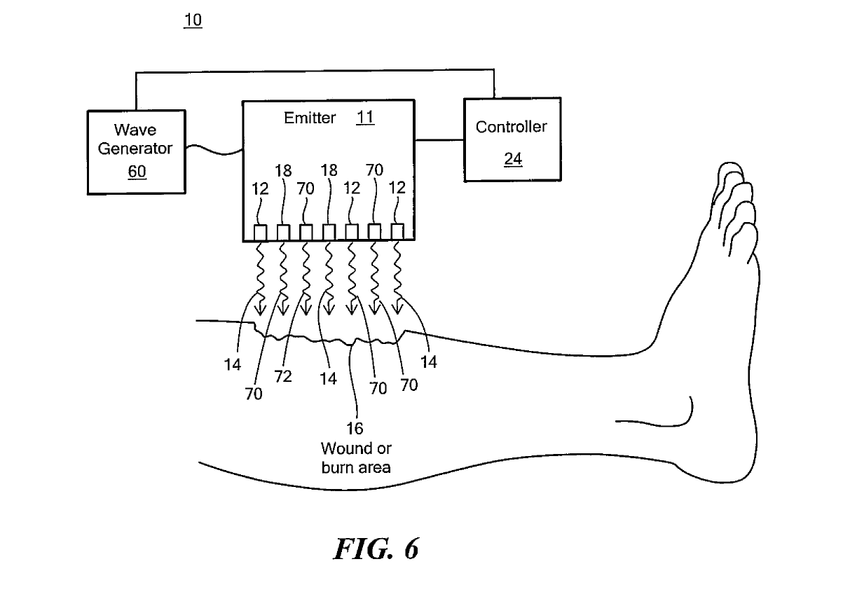

In one design, emitter 11, FIG. 6, where like parts have been given like numbers, may also include one or more red light sources 70 configured to emit red light 72 at a therapeutic energy level, e.g., 0.4 J/cm² to about 4 J/cm², to wound or burn area 16. Similar, as discussed above, controller 24 is coupled to emitter 11 having the one or more red light sources 70 and is configured to control the therapeutic energy level of red light 72 and one or more parameters associated with the red light, e.g., similar to the one or more parameters of the blue light and UVA light discussed above, to increase oxygenation, vascularization, and recruitment of immune cells in wound or burn area 16 and to enhance photoexcitation of accumulated intracellular chromophores and production of ROS and opportunistic pathogens. In one design, one or more red light sources 72 are preferably included in multi-emitter array 22, FIG. 2, as shown.

One example of the method for healing and disinfecting wounds of this invention includes applying blue light at a therapeutic energy level at a wound or burn area of a human or animal subject, step 100, FIG. 7. UVA light is applied at a therapeutic energy at a wound or burn area, step 102. The therapeutic energy of the blue light and the UVA light and one or more parameters associated with the blue light and the UVA light is controlled to product a photobiomodulation effect to induce a cytokine response in the cells of the wound or burn area to illicit recruitment and proliferation of cells to promote healing of the wound or burn area, step 104. In one example, the therapeutic energy level of the blue light and the UVA light and the one or more parameters associated with the blue light and the UVA light is controlled to activate photoexcitation of intracellular accumulated chromophores and the production of cytotoxic reactive oxygen species in the opportunistic pathogens such that a synergistic effect of the combination of the blue light at the therapeutic energy level and the UVA light at the therapeutic energy level kills opportunistic pathogens in the wound or burn area to disinfect an infected wound or burn area, step 106.

Blue light 14 applied to wound or burn area 16 at the therapeutic energy level and one or more parameters associated with blue light discussed above stimulates activation of keratinocytes. Activation of keratinocytes is required for production of collagen, a structural component of the epidermis and cytokine signaling to illicit an immune response which aids in the clearance of microbes.

Blue light 14 has not shown any inflammatory cell response and does not produce any significant change in p53 gene expression. This means blue light 14 may not be phototoxic to mammalian cells. An increase of p53 may lead to cell cycle arrest which may be useful in treating a skin cancer and increases further lead to cell apoptosis, (cellular death). Therefore, no increase to p53 expression from cells exposed to blue light 14 means blue light 14 does not contribute to cell death. Blue light 14 is non-cytotoxic and assists in cell signaling repair leading to regeneration and healing of damaged cells and tissue.

The antimicrobial effect of blue light 14 applied to wound or burn area 16 by system 10 and the method thereof, as discussed above, referred herein as blue light inactivation, may be provided by excitation of endogenous bacterial chromophores, such as porphyrins. The absorption of energy by the porphyrins leads to an excited energy state of the chromophore resulting in the production of a reactive oxygen species (ROS), most notably singlet oxygen. Additionally, photo sensitizers activated by the wavelengths of blue light 14 may be combined to enhance therapy.

In general, wound healing typically involves homeostasis, inflammation, granulation, fibrogenesis, re-epithelialization, neovascularization, and maturation contraction. Clinical trials using UVA light to promote wound healing have shown that UVA treatment is associated with an increase in production of MMP-1 and IFN-C production. These are associated with collagen production, stimulation of phagocytosis, promotion of lymphocytes, and a key component of innate and adaptive host immune functions thereby priming the immune system to attack foreign material such as host pathogens.

MMP-1 (Matrix metalloproteinase-1) is a collagenase encoded by humans. Collagenase cleaves proto-collagen to form the stable extracellular matrix of collagen supporting epithelia. Collagen is the main structural component of skin. IFN-C is an interferon. Interferons are members of a class of chemicals known as cytokines. Cytokines play a key role in cellular communication and serve to prime the immune system which aids in the clearance of foreign antigens such as those expressed by bacteria or mutagenized cells in the case of some cancers. Cytokines can initiate cellular repair.

UV light (specifically, UVA) has been proposed as a potential modulator of keratinocyte-melanocyte cross talk in promoting wound healing. Keratinocytes, the main cell type in the epidermis, form a self-renewing epithelial barrier to protect the skin against environmental hazards, while melanocytes, located in the basal layer of the epidermis, are dendritic-like pigment-producing cells, which protect keratinocytes against the DNA-damaging effects of UVB irradiation through production of melanin.

UVA light 20, unlike UVB and UVC, applied to wound or burn area 16 by system 10 and the method thereof at the therapeutic energy level and one or more parameters associated with UVA light 20 discussed above is generally well-tolerated by cells because it is weakly absorbed by DNA. This is because UVB is directly damaging DNA, whereas UVA excites endogenous chromophores, leading to the expression of ROS.

While all forms of UV radiation are potentially damaging the predominant form of naturally occurring damage is attributed to UVB. In contrast, UVA is associated with formation of (6-4) pyrimidine and pyrimidine photoproducts which are effectively repaired in human cells. DNA aside, another well-characterized photo damage signaling molecule is trans-urocanic acid (trans-UCA) which is isomerized to cis-UCA and exhibits potent immunosuppressive qualities through activation of Treg cells upon exposure to UVC light and UVB light, but not UVA light 20. This difference may be correlated to the relative protection of the cell nucleus to UVA damage, unlike UVB and UVC.

Therefore low doses of UVA light 20 applied to wound or burn area 16 at the therapeutic energy level and one or more parameters associated with UVA light by system 10 and the method thereof, discussed above may not be significantly cytotoxic and instead initiates a repair response will lead to cellular proliferation and regrowth of the affected tissue.

When wound or burn area 16 is exposed to UVA light 20 by system 10 and the method thereof, Eukaryotic Initiation Factor 2a subunit (eIF2a- Ser51) phosphorylation occurs and is implicated in cell proliferation and apoptosis, and elF2a-Ser51 executes a key translational control mechanism following UV irradiation that is dose and time-dependent. Low doses of UVA light 20 at the therapeutic energy level and one or more parameters associated with UVA light 20 promote tissue regeneration, instead of cellular death.

Keratinocytes cells make up about 95% of the cells of the epidermis (skin). Keratinocytes also serve as a scaffold to hold Langerhans cells and lymphocytes in place. In addition to providing a structural barrier, keratinocytes serve a chemical immune role as immunomodulators, responsible for secreting inhibitory cytokines in the absence of injury and stimulating inflammation and activating Langerhans cells in response to injury. Langerhans cells serve as antigen-presenting cells when there is a skin infection and are the first cells to process microbial antigens entering the body from a skin breach.

The antimicrobial properties of UVA Light 20 are similar to blue light 14 but with different absorption spectra on the chromophores.

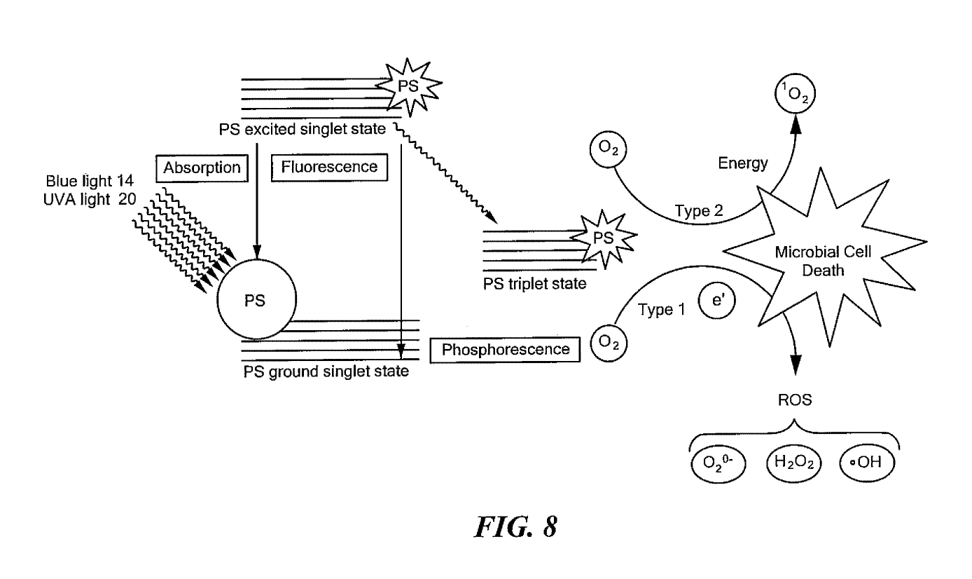

FIG. 8 shows an example of the process of photoinactivation by photoexcitation of endogenous chromophores by blue light 14 and UVA light 20 at their therapeutic energy levels and the one or more parameters associate with blue light 14 and UVA light 20 into reactive oxygen species (ROS).

The combination of the blue light 14 at the therapeutic energy level and UV light 20 at therapeutic energy level and the one or more parameters associated with the blue light 14 and UVA light 20 stimulates multiple signaling pathways of the host epithelia and cells of the immune system. Because system 10 and the method thereof combines blue light 14 and UV light 20 at the therapeutic energy level and the one or more parameters associated with the blue light 14 and UVA light 20, a healing and disinfection effect is achieved on the wound or burn area 16 such that the total result is not simple an additive function of one plus the other, but a synergistic effect. This is because when blue light 14 and UVA light 20 are administered in combination, a maximizing effect is achieved thereby multifold increasing cellular production of key chemicals and molecules leading to a robust effect.

Because the therapeutic dosage of the blue light 14 and the UVA light 20 is controlled and the damaging wavelengths of UVA and UVB light are omitted, the cells of wound or burn area 16 respond as though damage has occurred (since in nature UVA light 20 does not occur in the absence of UV-B). Essentially the process hijacks the cells defense response to photodamage in order initiate a robust cellular regeneration, proliferation, and immune response. Because this response is controlled and not found in nature it is most aptly defined herein as photobiomodulation—the use of light to elicit a predictable cellular response.

Thus, system 10 system and the method thereof for healing and/or disinfecting wounds and burns utilizes multiple wavelengths of blue light 14 and UVA light 20 delivered in various parameters, combinations and waveforms to control activation of multiple different cellular pathways that ultimately results in a maximized and cascade like healing function. This is because multiple cell types are activated, all of which individual serve to recruit repair and defense responses.

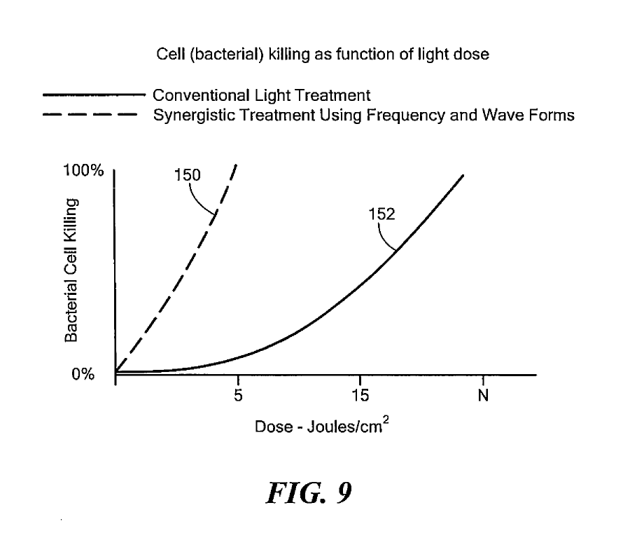

Because system 10 and the method thereof utilizes multiple pathways, lower doses of blue light 14 and UVA light 20 are required compared to one wavelength of light alone. This means controlled variable emitter 11 and multi-emitter array 22, FIGS. 1-3 and 5, with one more blue light sources 12 and one or more one or more UVA light sources 18 controlled by controller 24 can achieve efficacy in shorter time or with lower doses of blue light 14 and UVA light 20. Graph 150, FIG. 9, shows an example of the lower doses of blue light 14 and UVA light 20 provided by the synergistic effect of system 10 and the method thereof compared to conventional treatment, indicated by graph 152. This feature may reduce the costs of the components of system 10.

As discussed above, controller 24 is preferably configured to variably control emitter 11 and multi-emitter array 22 such that the therapeutic energy level of the dosage or amount of blue light 14 and UVA light 20 are collectively reduced. This is a key feature of system 10 because there is a threshold where the therapeutic energy level of blue light 14 and UVA light 20 may be too high and damage to the cells of the epithelia, as known by those skilled in the art. There is also a level of blue light 14 an UVA light 20 at the therapeutic energy level that may be too low and may have an insufficient effect on reducing the pathogen load, e.g., 0.01 J per cm². Therefore, system 10 provides a synergistic response in both wound healing and pathogen killing.

Moreover, by selecting blue light 14 and UVA light 20 and omitting harmful UVB and UVC, which are typically produced in a conventional broad band UV system, up to about 90% of the damaging component of UV light is removed. This allows for a greater dose of UVA light 20 and blue light 14 to be administered on wound or burn area 16 without causing unwanted cellular damage. Additionally, blue light 14 and UVA light 20 are is well tolerated by the patient or animals.

The antimicrobial effect of blue light 14 is oxygen dependent. Thus, increased oxygenation using red light 70, FIG. 6, leads to an enhanced effect of blue light 14 and UVA light 20. Because pigmentation is associated with virulence, more virulent microbes, e.g., greater pigmentation, are at greater susceptibility to inactivation with the addition of red light 72 which means that any surviving microorganisms are anticipated to be more susceptible to antibiotic treatment. Thus, system 10 and the method thereof provides an additional way to control the bacterial load in wound and burn area 16 leading to the body's ability to heal naturally.

The pigments of the pathogen (virulence factors) under normal conditions serve to scavenge free radicals, which is a microbial defense response to the immune system of the host. However, the pigments are illicitly turned into agents of the pathogens own destruction thereby toxifying them directly and making them more susceptible to the hosts own inactivation mechanisms (peroxide attack through lysosome digestion).

The healing and regenerative properties of red light 72 may include an increased circulation and formation of new capillaries (blood flow provides additional oxidation and nutrients to the damaged region of tissue) allowing for an increase in phagocytes (white blood cells) and components of both the innate and adaptive immune systems. White blood cells, for example, digest bacteria thereby decreasing the presence of toxins while also removing dead or damaged host cells which bacteria would otherwise consume as nutrients to further their own proliferation. This leads to a reduction of inflammation. Red light 72 stimulates the lymphatic system and reduces lymphedema. Red light 72 also stimulates proliferation of fibroblasts which synthesize collagen, elastin, and proteoglycans, all of which are critical to the healing process. The production of collagen leads to wound closure. Red light 72 also stimulates tissue granulation which allows for new connective tissue and vascularization at the surface of the wound.

The antimicrobial mechanism of red light 72 is the same blue light 14 and UVA light 20 discussed above, e.g., photoexcitation of endogenous chromophores, such as protoporphyrin IX. Porphyrins are a group of heterocyclic macrocycle organic compounds, composed of four modified pyrrole subunits interconnected at their α carbon atoms via methine bridges. In addition to photoexcitation, red light 72 stimulates the immune system, the natural defense system of the host against pathogens.

Additionally, photosensitizers (PS) activated by at the wavelengths of red light 72 discussed above may be combined to enhance therapy, e.g., Toluidine Blue O. PS are a chemical agents that act similarly to chromophores but are not endogenously produced by the cell, typically these are dyes. When exposed to some wavelength of light these chemicals become photoexcited resulting in produce of ROS which are toxic to the pathogen.

The result is system 10 and method for healing and/or disinfecting wounds and burns of one or more embodiments of this invention controls the application of blue light 14, UVA light 20 and/or red light 72 to wound or burn area 16 to initiate a cellular response that tricks the cell into responding to a photodamaging event that is largely ameliorated and such a combinatorial treatment represents a photobiomodulation approach. The body initiates a cytokine mediated repair response in the presence of UVA light 20, without the damaging effects of UVB or UVC light. Thus, system 10 and the method thereof provides a treatment that heals and/or disinfects wounds that initiates cells, including chronic non-healing cells, to execute various molecular pathways that lead to recruitment of healthy cells, such as keratinocytes, that will subsequently populate, regrow, and repair wound or burn area 16. The cells of the wound or burn area 16 also initiate a series of repair mechanisms. Because significant photodamage is unlikely to occur, the damaged cells can repair. In cases where repair is not possible, the cellular response will lead to apoptosis, a controlled form of cell death. At the same time, recruitment of adaptive immunity components (macrophages, T-cells) occur at the wound or burn area 16 leading to the removal of dead cellular material, debris, and initiate destruction of pathogens by phagocytosis. These series of events, ultimately will also facilitate reducing inflammation of the wound area.

System 10 and method for healing and/or disinfecting wounds and burns provides a multi-modal technology for disinfecting pathogens present on wound bed or burn area 16 using phototoxic by-products and coupling their destruction by cells of the host immune system. At the same time, system 10 and the method thereof elicits a cellular driven mechanism to repair of damaged host cells and recruitment of keratinocytes to produce collagen and regrow the wound area. Treatment of infected non healing wounds using system 10 and the method thereof provides an enhanced or synergistic response that effectively and efficiently provides disinfection and wound healing. Thus, system 10 provides fast healing when compared to untreated wounds and wounds or burns treated only with conventional antimicrobial modalities. System 10 and the method thereof provides for simultaneously killing drug resistant pathogens present on the wound or burn area 16 and healing wound or burn area 16. System 10 is less complex than conventional systems and methods and can easily be used in many different physical environments, including places that antibiotics/antimicrobials are not readily accessible. The result is, system 10 is highly effective, versatile, and cost efficient.

Although specific features of the invention are shown in some drawings and not in others, this is for convenience only as each feature may be combined with any or all of the other features in accordance with the invention. The words “including”, “comprising”, “having”, and “with” as used herein are to be interpreted broadly and comprehensively and are not limited to any physical interconnection. Moreover, any embodiments disclosed in the subject application are not to be taken as the only possible embodiments.

In addition, any amendment presented during the prosecution of the patent application for this patent is not a disclaimer of any claim element presented in the application as filed: those skilled in the art cannot reasonably be expected to draft a claim that would literally encompass all possible equivalents, many equivalents will be unforeseeable at the time of the amendment and are beyond a fair interpretation of what is to be surrendered (if anything), the rationale underlying the amendment may bear no more than a tangential relation to many equivalents, and/or there are many other reasons the applicant cannot be expected to describe certain insubstantial substitutes for any claim element amended.

Other embodiments will occur to those skilled in the art and are within the following claims.

What is claimed is:

1. A system for healing and/or disinfecting wounds and burns, the system comprising:
an emitter including:
one or more blue light sources configured to emit blue light at a therapeutic energy level at a wound or burn area of a human or animal subject, and
one or more ultraviolet-A (UVA) light sources configured to emit UVA light at a therapeutic energy level at the wound or burn area; and
a controller coupled to the emitter configured to control the therapeutic energy level of the blue light and the UVA light and one or more parameters associated with the blue light and the UVA light to produce a photobiomodulation effect in order to induce a cytokine response in cells of the wound or burn area to elicit recruitment and proliferation of cells to promote healing of the wound or burn area.

2. The system of claim 1 in which the controller is configured to control the therapeutic energy level of the blue light and the UVA light and one or more parameters associated with the blue light and the UVA light to activate photoexcitation of intracellular accumulated chromophores and production of cytotoxic reactive oxygen species in opportunistic pathogens such that a synergistic effect of the combination of the blue light at the therapeutic energy level and the UVA light at the therapeutic energy level and the one or more parameters associated with the blue light and the UVA light kills opportunistic pathogens in the wound or burn area to disinfect an infected wound area or an infected burn area.

3. The system of claim 1 in which the therapeutic energy level of the blue light and the UVA light is in the range of about 0.4 J/cm² to about 4 J/cm².

4. The system of claim 1 in which the one or more parameters of the blue light and the UVA light controlled by the controller include one or more of: a wavelength of the blue light, a wavelength of the UVA light, a frequency of the blue light, a frequency of the UVA light, one or more waveforms of the blue light, one or more waveforms of the UVA light, one or more duty cycles of the blue light, and/or one or more duty cycles of the UVA light.

5. The system of claim 4 in which the wavelength of the blue light is in the range of about 405 nm to about 470 nm.

6. The system of claim 4 in which the wavelength of the UVA light is in the range of about 315 nm to about 400 nm.

7. The system of claim 4 in which the frequency of the blue light is in the range of about 0.5 Hz to about 1,000 Hz.

8. The system of claim 4 in which the frequency of the UVA light is in the range of about 0.5 Hz to about 1,000 Hz.

9. The system of claim 4 in which the one or more waveforms include one or more of a sinewave, a square wave, a triangle wave, and/or a sawtooth wave.

10. The system of claim 1 in which the controller is configured to control the one or more blue light sources and the one or more UVA light sources to emit the blue light and the UVA light as pulsed light.

11. The system of claim 1 in which the controller is configured to control the one or more blue light sources and the one or more UVA light sources to emit the blue light and the UVA light as continuous light.

12. The system of claim 1 in which the controller is configured to control the one or more blue light sources and the one or more UVA light sources the blue light and the UVA light in a combination of pulsed light and continuous light.

13. The system of claim 10 in which the one or more duty cycles of the blue light includes one or more of: a 25% duty cycle, a 50% duty cycle, or a 75% duty cycle.

14. The system of claim 10 in which the one or more duty cycles of the UVA light includes one or more of: a 25% duty cycle, a 50% duty cycle, or a 75% duty cycle.

15. The system of claim 1 in which the one or more blue light sources and the one or more UVA light sources are configured as a multi-emitter array.

16. The system of claim 15 in which the controller is configured to control the multi-emitter array to provide the blue light at the therapeutic energy level and the UVA light at the therapeutic energy level and at the one or more parameters associated with the blue light and the UVA light.

17. The system of claim 1 in which the controller is configured to shut off the one or more blue light sources and the one or more UVA sources when a predetermined therapeutic dosage of blue light and UVA light is applied to the wound or burn area.

18. The system of claim 1 in which the emitter includes one or more red light sources configured to emit red light at a therapeutic energy level at the wound or burn area.

19. The system of claim 18 in which the controller is coupled to the one or more red light source and is configured to control the therapeutic energy level of the red light and one or more parameters associated with the red light to increase oxygenation, vascularization and recruitment of immune cells in the wound or burn area and to enhance the photoexcitation of accumulated intracellular chromophores and production of cytotoxic reactive oxygen species in opportunistic pathogens.

20. The system of claim 19 in which the one or more blue light sources, the one or more UVA light sources, and the one or more red light sources are configured as a multi-emitter array.

21. The system of claim 20 in which the controller is configured to control the multi-emitter array to provide the blue light at the therapeutic energy level, the UVA light at the therapeutic energy level, and the red light at the therapeutic energy level, and one or more parameters associated with the blue light, the UVA light, and the red light.

22. The system of claim 1 in which the controller is responsive to a wound or burn area detection device and is configured to determine a size of the wound or burn area.

23. The system of claim 1 in which the controller is configured to provide the therapeutic energy level of the blue light, the therapeutic energy level of the UVA light, and the one or more parameters associated with the blue light and the UVA light by controlling the power applied to the emitter.

24. The system of claim 1 further including a display device coupled to the controller.

25. A system for healing and/or disinfecting wounds and burns, the system comprising:
an emitter including:
one or more blue light sources configured to emit blue light at a therapeutic energy level at a wound or burn area of a human or animal subject, and
one or more (ultraviolet) UVA light sources configured to emit UVA light at a therapeutic energy level at the wound or burn area; and
a controller coupled to the one or more blue light sources and the one or more UVA light sources configured to control the therapeutic energy level of the blue light and the UVA light and one or more parameters associated with the blue light and the UVA light to activate photo excitation of intracellular accumulated chromophores in the production of cytotoxic reactive oxygen species in opportunistic pathogens such that a synergistic effect of the combination of the blue light at the therapeutic energy level and the UVA light at the therapeutic energy level and the UVA light at the therapeutic energy level and the one or more parameters associated with the blue light and the UVA light kills the opportunistic pathogens in the wound or burn area to disinfect an infected wound or an infected burn area.

26. A system for healing and/or disinfecting wounds and burns, the system comprising:
an emitter including:
one or more blue light sources configured to emit blue light at a therapeutic energy level at a wound or burn area of a human or animal subject, and
one or more (ultraviolet) UVA light sources configured to emit UVA light at a therapeutic energy level at the wound or burn area; and
a controller coupled to the one or more blue light sources and the one or more UVA light sources configured to control the therapeutic energy level of the blue light and the UVA light and one or more parameters associated with the blue light and the UVA light to produce a photobiomodulation effect in order to induce a cytokine response in cells of the wound or burn area to elicit recruitment and proliferation of cells to promote healing of the wound or burn area and to activate photoexcitation of intracellular accumulated chromophores and production of cytotoxic reactive oxygen species in opportunistic pathogens such that a synergistic effect of the combination of blue light at the therapeutic energy level and UVA light at the therapeutic energy level and the UVA light at the therapeutic energy level and the one or more parameters associated with the blue light and the UVA light kills the opportunistic pathogens in the wound or burn area to disinfect an infected wound area or an infected burn area.

27. A method for healing and/or disinfecting wounds and burns, the method comprising:
applying blue light at a therapeutic energy level at a wound or burn area of a human or animal subject;
applying ultraviolet-A (UVA) light at a therapeutic energy level at the wound or burn area; and
controlling the therapeutic energy level of the blue light and the UVA light and one or more parameters associated with the blue light and the UVA light to produce a photobiomodulation effect in order to induce a cyctokine response in the cells of the wound or burn area to illicit recruitment and proliferation of cells to promote healing of the wound or burn area.

28. The method of claim 27 in which controlling the therapeutic energy level of the blue light and the UVA light and the one or more parameters associated with the blue light and the UVA light activates photo excitation of intracellular accumulated chromophores and the production of cytotoxic reactive oxygen species in the opportunistic pathogens such that a synergistic effect of the combination of the blue light at the therapeutic energy level and the UVA light at the therapeutic energy level and the UVA light at the therapeutic energy level and the one or more parameters associated with the blue light and the UVA light kills opportunistic pathogens in the wound or burn area to disinfect an infected wound area or an infected burn area.

29. The method of claim 27 in which controlling the one or more parameters of the blue light and the UVA light includes controlling one or more of: a wavelength of the blue light, a wavelength of the UVA light, a frequency of the blue light, a frequency of the UVA light, one or more waveforms of the blue light, one or more waveforms of the UVA light, one or more duty cycles of the blue light, and one or more duty cycles of the UVA light.

30. The method of claim 27 in which the blue light and the UVA light are applied at a therapeutic energy level in the range of about 0.4 J/cm² to about 4 J/cm².

31. The method of claim 29 in which the blue light is applied at a wavelength in the range of about 405 nm to about 470 nm.

32. The method of claim 29 in which the UVA light is applied at a wavelength in the range of about 315 nm to about 400 nm.

33. The method of claim 29 in which the one or more waveforms include a sinewave, square wave, a triangle wave, and/or a sawtooth wave.

34. The method of claim 29 in which the blue light is applied at a frequency in the range of about 0.5 Hz to about 1,000 Hz.

35. The method of claim 29 in which the UVA light is applied at a frequency in the range of about 0.5 Hz to about 1,000 Hz.

36. The method of claim 29 in which the one or more duty cycles of the blue light include one or more of: 25%, 50%, or 75% duty cycle.

37. The method of claim 29 in which the one or more duty cycles of the UVA light includes one or more of: 25%, 50%, or 75% duty cycle.

38. The method of claim 27 further including applying red light at a therapeutic energy level and one or more parameters associated with the red light at the wound or burn area.

39. The method of claim 38 further applying red light at a therapeutic energy level and one or more parameters associated with the red light at the wound or burn area to increase vascularization and recruitment of immune cells of the wound area and oxygenation in the cells of the wound or burn area to enhance the photobiomodulation and/or the photo excitation of intracellular chromophores and production of cytotoxic reactive oxygen species in opportunistic pathogens.

40. The method of claim 27 in which the therapeutic energy level of the blue light, the therapeutic energy level of the UVA light, and the one or more parameters associated with the one blue light and the UVA light is controlled by adjusting a power level applied to one or more blue light sources and one or more UVA light sources.
